(19) United States
(12) Patent Application Publication
(10) Pub. No.: US 2020/0121479 A1
(43) Pub. Date: Apr. 23, 2020

(54) HAND STRENGTH ENHANCEMENT GLOVE

(71) Applicant: Ansell Limited, Richmond, Victoria (AU)

(72) Inventors: Eric Michael Thompson, Central, SC (US); Carrie Lirae Merck, Taylors, SC (US); Mathotarallage Amani Rasika Mathota, Kiribathgoda, Kelaniya (LK); Norberto Hector Perales Solis, Ciudad Juarez (MX); Adrian Fernando Sanchez Torres, Chihuahua (MX); Alejandra Estrada Meza, Chihuahua (MX)

(21) Appl. No.: 16/628,146

(22) PCT Filed: Jul. 11, 2018
(86) PCT No.: PCT/AU2018/000114

§ 371 Date: Jan. 2, 2020

Related U.S. Application Data
(60) Provisional application No. 62/531,096, filed on Jul. 11, 2017.

(51) Int. Cl. A61F 2/70; A41D 19/00; A41D 19/015; A61F 2/58; A61B 5/00

(52) U.S. Cl. CPC A61F 2/70; A41D 19/0006; A41D 19/01582; A61F 2002/587; A61F 2/586; A61B 5/6806; A41D 19/0024

(57) ABSTRACT

A glove having tendons, substantially as shown and described in connection with at least one of the figures, as set forth more completely in the claims, is provided. In some embodiments, a glove system capable of assisting a user may include a first knitted glove having at least one finger or thumb having at least one force detecting sensor and a cuff, at least one tendon adhered to a portion of at least one finger or thumb and traversing a length of the at least one finger or thumb to the cuff, at least one actuator attached to the at least one tendon, and at least one controller in electrical communication with the at least one force detecting sensor and the at least one actuator, wherein the at least one tendon undergoes a tensile force pulling the at least one finger or thumb in a closed position upon detection of a force by the at least one force detecting sensor.

### BACKGROUND

### Field of the Invention

Embodiments of the present invention relate generally to protective articles and, more particularly, to glove systems capable of assisting tendons.

### Description of the Related Art

Gloves are used in many industries and in households. Many activities are of a repetitive nature, which can cause or exacerbate repetitive motion injuries, such as lateral epicondylitis and carpal tunnel syndrome and musculo-skeletal disease. Also, the longer a person engages in activities using the hand, the more tired the hand can become. The gripping power of gloves can aid users to cope with these problems. For example, gloves having various textures have been manufactured, which improve oil, dry, and/or wet gripping properties. And, gloves have been made to assist users, i.e., a force is provided to a glove to close and keep closed the hand during activities.

Previous assistive gloves are cut-and-sewn, leather gloves that have seams, which can make the gloves less flexible and more bulky, restricting the movement of fingers and increasing the force needed to close the hand. Other assistive gloves have included metal and/or thick plastic members that are actuated electrically to open and close the hand. Workers also complain that such gloves are too bulky and inflexible for the intricate work that they perform. Furthermore, past assistive gloves are expensive and it is therefore favorable to protect such gloves from contact with chemicals and other environmental factors that degrade the glove.

With the foregoing in view, comfortable, thin, flexible gloves comprising a knitted liner, optionally seamless knitted liners, having tendons capable of flexing a hand, which are easy and inexpensive to manufacture, represent a significant advance in the art.

### SUMMARY

A glove having tendons, substantially as shown and described in connection with at least one of the figures, as set forth more completely in the claims, is provided. Various advantages, aspects and novel and inventive features of the present disclosure, as well as details of illustrative embodiments thereof, will be more fully understood from the following description and drawings. The foregoing summary is not intended, and should not be contemplated, to describe each embodiment or every implementation of the embodiments. Other and further embodiments of the present disclosure are described below.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1A depicts a palm side of a thin, flexible, knitted left-handed inner glove having tendons, according to some embodiments of the disclosure;

FIG. 1B depicts a palm side of a thin, flexible, knitted left-handed inner glove having tendons, according to some embodiments of the disclosure;

FIG. 2 depicts a backhand side of a thin, flexible, knitted right-handed inner glove having tendons, according to some embodiments of the disclosure;

FIG. 3A and FIG. 3B depict palm and backhand views of an outer right-handed glove, in accordance with some embodiments of the disclosure;

FIGS. 4A and 4B depict the palm and backhand views of FIG. 1A and FIG. 2, respectively, along with a robotic sleeve portion attached to the cuff, in accordance with some embodiments of the disclosure;

FIG. 5 depicts a palm side of a thin, flexible, knitted left-handed inner glove having a modified cuff and tendons, according to some embodiments of the disclosure;

FIG. 6 depicts a palm side of a thin, flexible, knitted left-handed inner glove having a modified cuff, tendons, and a stabilizer having tunnels, according to some embodiments of the disclosure; and

To facilitate understanding, identical reference numerals have been used to designate comparable elements that are common to the figures. The figures are not necessarily drawn to scale and may be simplified for clarity. It is contemplated that elements and features of one embodiment may be beneficially incorporated in other embodiments without further recitation. Other and further embodiments of the present disclosure are described below.

### DETAILED DESCRIPTION

Before describing embodiments of the present invention in detail, the terminology used herein is for the purpose of describing particular embodiments only, and is not intended to be limiting. The invention should not necessarily be limited to specific compositions, materials, designs or equipment, as such may vary. All technical and scientific terms used herein have the usual meaning that is conventionally understood by persons skilled in the art to which embodiments of this invention pertain, unless context defines otherwise. Also, as used in this specification and the appended claims, the singular forms “a,” “an,” and “the” may include plural referents unless the context clearly dictates otherwise.

Embodiments of the present disclosure may advantageously be used to assist users in gripping tools and other objects by applying additional force(s) provided by embodiments of the glove having tendons. Embodiments of the present disclosure may advantageously be used to prevent injuries, including injuries because of repetitive motions. Embodiments of the present disclosure may advantageously be used to enhance productivity and ergonomics. Embodiments of the present disclosure may advantageously be used to protect parts of equipment and systems comprising embodiments described herein.

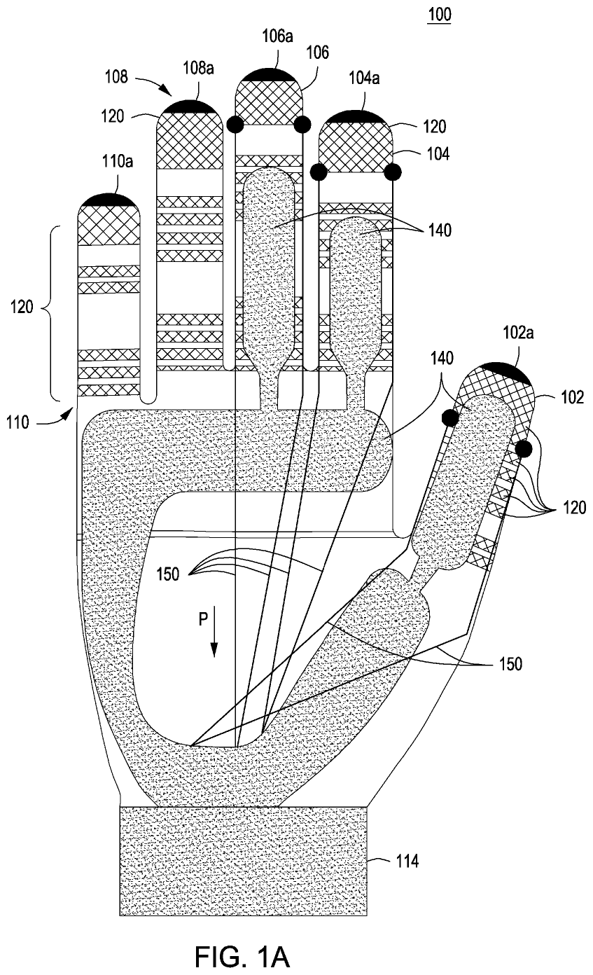

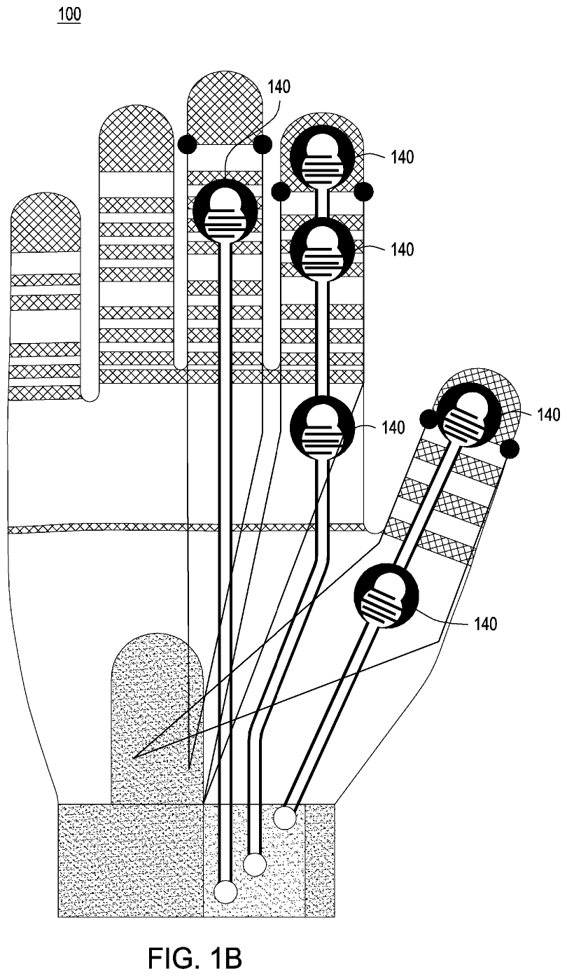

FIG. 1 depicts a palm side of a thin, flexible, knitted left-handed inner glove 100 having tendons 150, according to some embodiments of the disclosure. As seen in FIG. 1, the glove 100 is a left-handed inner glove in which the palm 112 is displayed. The glove 100 includes a thumb 102, an index finger 104, a middle finger 106, a ring finger 108, a little finger 110, a cuff 114, and an opening 116 capable of receiving the hand of a wearer. Each of the thumb 102, the index finger 104, the middle finger 106, the ring finger 108, and the little finger 110 further comprises tips at distal ends, a thumbtip 102a, an index fingertip 104a, a middle fingertip 106a, a ring fingertip 108a, and a little fingertip 110a. The glove 100 also comprises tendons 150. As shown, the thumb 102 and the index finger 104 each comprise the tendon 150. In practice, there can be a tendon 150 in each of the thumb 102, and the fingers 104, 106, 108, and 110 or any combination thereof. In some embodiments, for example, there may be more than one tendon 150 in any finger 104, 106, 108, and 110 or more than one tendon 150 in the thumb 102. The tendons 150 can be a yarn, a string, a polymer, a wire, a fiber, or any monofilament. In at least one embodiment, the tendon 150 is a filament comprising, for example, a high performance polyethylene (HPPE) filament. Also, in at least one embodiment, the tendon 150 is a braided HPPE filament, which optionally further comprises a coating, such as a polytetrafluoroethylene coating or a silicon coating. In practice, many knitted gloves marketed by Ansell Ltd. can have the tendons 150 added to manufacture the glove 100. For example, either of models GT3279 or GT3280 would be suitable for the inner glove 100. At least one other exemplary inner glove 100 is model GT3287 or model GT3440. Also, at least one inner glove 100 comprises a hooks and loops fastener strap 146 for securing the inner glove 100 to an outer glove, as described more fully below.

The glove 100 also depicts borders 120. The border 120 roughly correlates to the position of a knuckle on a human hand. As shown, the tendons 150 are adhered or otherwise tied to a fingertip or thumbtip, such as thumbtip 102a and the index fingertip 104a. In some embodiments, the tendons 150 are sewn (e.g., chain stitched) and/or knotted to the zones/borders 120. As shown on the index finger 104, the tendon 150 travels outside the index finger 104, enters through the knitted structure of the index finger 104 at insertion point 160, wherein the border 120 is between the knot 130 and the insertion point 160. The tendon 150 stays inside the index finger 104 before exiting at exit point 162. The tendon 150 then re-enters the index finger 104 at a second insertion point 164 and re-exits at a second exit point 166. As shown, the tendons 150 traverse a lateral perimeter 170 of the index finger 104. Also, as shown, the tendon 150 on the index finger 104 enters the glove 100 twice—at the insertion point 160 and at the second insertion point 164 although, in practice, a plurality of insertion points are possible. As will be described more fully below, providing a pulling force P at inflection point 180 of the tendon 150 causes a bending of the index finger 104. Additionally, the tendons are optionally inserted into palm areas, and/or inserted into a floating thumb strap. In some embodiments, the tendons 150 may be attached by sewing channels directly onto the glove 100, on palm or back of hand, and/or lacing the tendons 150 between the glove 100 and the sewn channel thread.

In some embodiments, the borders 120 can be viewed as having no structure different than the glove 100 and exists only to show the relative positioning of the tendons 150. Alternately, the borders 120 may comprise fortified areas, e.g., reinforcements, for example, reinforcements having variable knit plaiting yarns to strengthen the areas around the ingress and egress of the tendons 150 into the glove 100. In some embodiments, the borders 120 can be reinforcements made of patches of material. In some embodiments the borders 120 may be made by Knitted Variable Stitch Dimension (KVSD). For example, the variable stitch dimension is achieved by one or more of 1) varying the depth of penetration of the knitting needle into fabric being knitted by a computer program, 2) adjusting the tension of yarn between a pinch roll and knitting head by a mechanism controlled by a computer and 3) casting off or picking up additional stitches in a course, as is described in U.S. Pat. No. 7,434,422, which is commonly assigned and incorporated by reference in its entirety. An inner glove 100 may also comprise a knitted layer having two or more yarns, seamless knit technology according to the co-pending, commonly assigned U.S. Patent Publ. Nos. 2010/0275341 and 2010/0275342, each of which is herein incorporated by reference in its entirety. The inner glove 100 may also comprise a knitted layer incorporating Automated Knitted Liner (AKL) technologies, developed by Ansell Limited. The inner glove 100 may also comprise a knitted layer having two or more yarns having variable plaiting, as is known to those in the art, to make an inner glove 100 having borders 120. At least one embodiment according to the present disclosure includes at least one of three ridge reinforced gloves, light duty, medium duty, and heavy duty gloves, as disclosed in US Publ. No. 2013/0205469, which is commonly assigned and incorporated herein by reference in its entirety. Those ridge-reinforced gloves comprise ridges. Accordingly, the ridge can be used as the borders 120 and the ridge-reinforced gloves can be used as the inner glove 100.

The thin, flexible, seamless, knitted gloves 100 can be made from a yarn having one of various deniers and using 13- or 15- or 18-knit gauges, and can be comprised of such materials as nylons, KEVLAR®, p-aramids, NOMEX®, m-aramids, SPECTRA®, DYNEEMA®, ultra-high molecular weight polyethylene, TSUNOOGA®, SPANDEX®, LYCRA®, elastane yarns, polyesters, rayon, cotton, fiberglass, and the like, and blends of the foregoing. In some embodiments, yarns comprising these materials may be treated with chemicals to impart other desirable properties, such as flame- and/or heat-resistance. Also, comfort, moisture control and absorbance, scratch resistance, cushioning, strength, and the like can be imparted to knitted liners by plaiting various yarns into the knitted glove. Furthermore, the knitted liners may comprise separately knitted sections and/or KVSD, the use of which allows relatively dense, knitted conductive sections at finger and thumb tips, facilitating their effectiveness with capacitive touchscreens, which, in some applications, may be more effective than plaiting a conductive yarn within the fingertips because of a greater number of conductive stitches per inch. Such technologies are disclosed in commonly assigned U.S. Pat. No. 6,962,064 and co-pending, commonly assigned U.S. Patent Publ. No. 2009/0211305, which are incorporated herein by reference in their entireties.

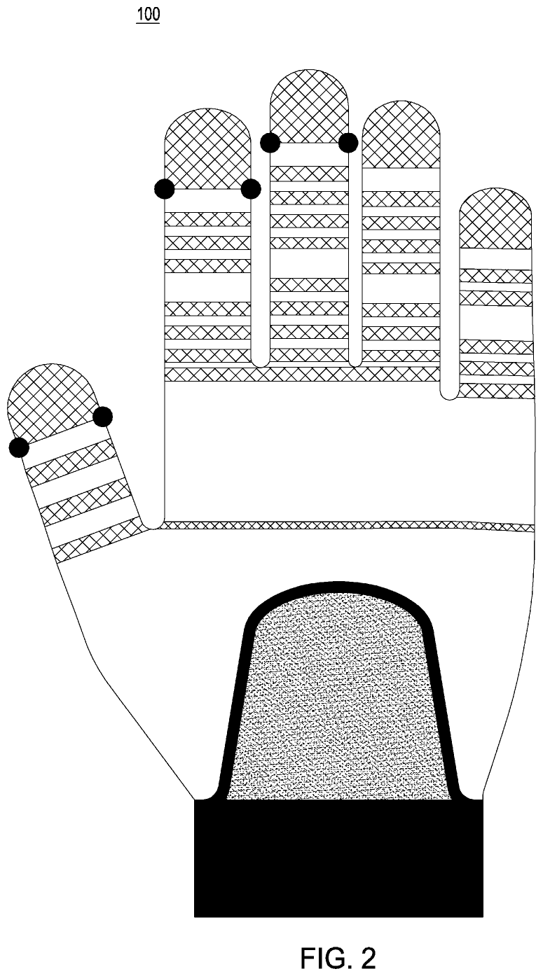

FIG. 2 depicts a backhand side of a thin, flexible, knitted right-handed inner glove 200 having tendons 150, according to some embodiments of the disclosure. As seen in FIG. 2, the glove 200 is a right-handed inner glove in which the backhand side 212 is displayed. The glove 200 includes a thumb 102, an index finger 104, a middle finger 106, a ring finger 108, a little finger 110, a cuff 114, and, optionally, a bead 118 for assisting the wearer to don the glove 200. Each of the thumb 102, the index finger 104, the middle finger 106, the ring finger 108, and the little finger 110 further comprises tips at distal ends, a thumbtip 102a, an index fingertip 104a, a middle fingertip 106a, a ring fingertip 108a, and a little fingertip 110a. The glove 200 also comprises tendons 150. In some embodiments, there may be more than one tendon 150 in any finger 104, 106, 108, and 110 or more than one tendon 150 in the thumb 102. The tendons 150 can be a yarn, a string, a polymer, a wire, a fiber, or any monofilament. In at least one embodiment, the tendon 150 is a monofilament comprising nylon materials, such as nylon 6, nylon 6,6, or nylon 4,6. In at least one embodiment, the tendon 150 is a monofilament comprising polyvinylidene fluoride (PVDF). In at least one embodiment, the tendon 150 is a filament comprising polyethylene, such as an ultra-high molecular weight polyethylene (UHMWPE), marketed as DACRON®, SPECTRA® or DYNEEMA®. In at least one embodiment, the tendon 150 comprises an HPPE material, as discussed above. Also, at least one embodiment comprises a braid of the HPPE materials described above or braids combining any of the filaments or monofilaments described above.

In operation, the tendons 150 would be positioned on the palm side so that when a pulling force is applied, as described above, the fingers and/or thumb of a wearer become closed. One or more of the power actuators described below are connected with one or more tendons 150 in one or more of the thumb 102, the index finger 104, the middle finger 106, the ring finger 108, and the little finger 110, respectively. It is to be understood that the power actuators can also be employed on the inner glove 100.

In some embodiments, the inner glove 200 comprises elastic yarns, such as SPANDEX® or LYCRA®, for promoting the stretchability of the inner glove 200.

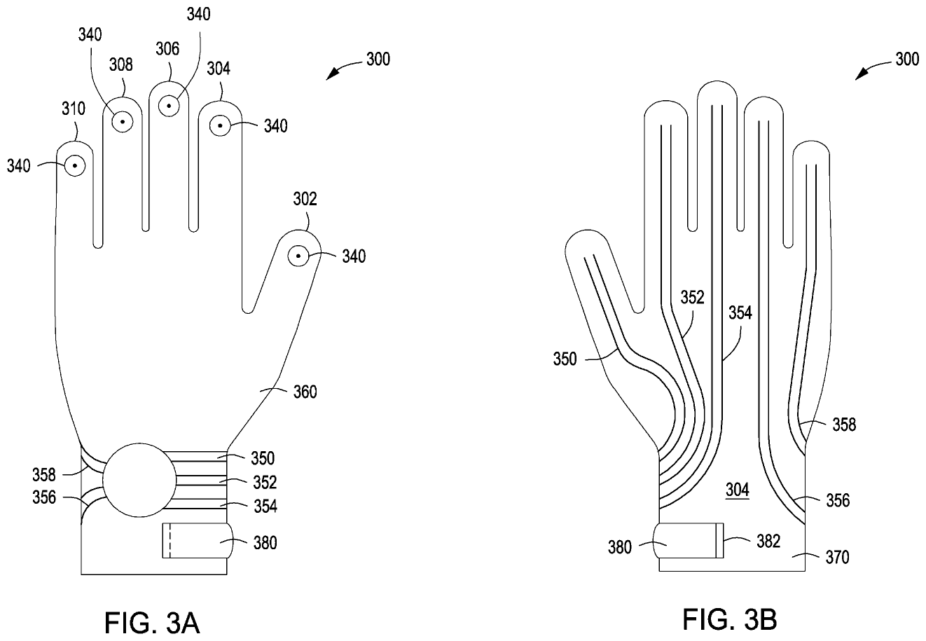

FIG. 3A and FIG. 3B depict an inner right-handed glove 300, in accordance with some embodiments of the disclosure. The inner right-handed glove 300 will be sized to fit with an outer glove, discussed below (and/or a middle glove, as discussed below), to protect the inner glove 300 (or middle glove) from wear. FIG. 3A depicts a palm side 360 of the inner right-handed glove 300. The inner glove 300 comprises force detecting sensors 340 on a thumbtip 302, an index fingertip 304, a middle fingertip 306, a ring fingertip 308, and a little fingertip 310. The force detecting sensors 340 are in electrical communication with conductive members 350, 352, 354, 356, 358, which are in electrical communication with a microprocessor, which calculates a force(s) needed to be applied an relates the force(s) to the power actuators and a power supply (not shown). The conductive members 350, 352, 354, 356, 358 may comprise conductive threads, conductive inks, wires, or, as described below, conductive yarns, etc., as are known to those in the art. In some embodiments, the force detecting sensors 340 are strain gauges. In some embodiments, the force detecting sensors 340 are non-stretchable. In some embodiments, the force detecting sensors 340 are stretchable and are encapsulated within various polymeric layers, as described below. Suitable stretchable sensors are supplied by, for example, Tacterion GmbH of Germany, DuPont of Wilmington, Del. and others. Encapsulating the force detecting sensors advantageously protects the sensors against water/moisture damage.

FIG. 3B depicts a backhand side 370 of the inner right-handed glove 300. The inner glove 300 comprises force detecting sensors 340 on a thumbtip 302, an index fingertip 304, a middle fingertip 306, a ring fingertip 308, and a little fingertip 310. The force detecting sensors 340 are in electrical communication with conductive members 350, 352, 354, 356, 358. The conductive members 350, 352, 354, 356, 358 may comprise conductive threads, wires, etc., as are known to those in the art. In some embodiments, the inner right-handed glove 300 may comprise a strap 380, wherein the strap 380 (located on the outer glove) exits from the strap outlet and returns into the outer right-handed glove. In some embodiments, the strap 380 can releasably engage the right-handed outer glove with the inner glove 100, 200, 300. Also, because the inner gloves 100, 200, 300 comprise a knitted structure, the hooks of the strap 380 can engage loops that are inherently part of the knitted structure at a distal end 382. In at least one embodiment, at least one patch (not shown) comprising hooks (of a hooks and loops fastener) is disposed on an internal part of the inner glove 300, wherein the hooks are capable of releasably adhering the inner glove 300 with the loops of the knitted structure of an outer glove. Furthermore, embodiments of the disclosure include an inner glove 300 (or any inner glove disclosed herein) having tendons 150 attached to every fingertip 304, 306, 308, 310 and thumbtip 302 but wherein the force detecting sensors 340 are only present on, for example, two fingertips 306, 308 and the thumbtip 302. An outer glove (not shown), can be any glove to protect the inner glove 100, 200, 300 or any other inner glove disclosed herein. At least two suitable outer gloves are glove model no. 11-840 and 11-800, manufactured by Ansell Limited, which are incorporated by reference in entirety.

Each of the thumb 302, the index finger 304, the middle finger 306, the ring finger 308, and the little finger 310 may optionally comprise conductive yarns and/or conductive additives. Conductive yarns can be used to electrically connect the force detecting sensors 340 with the microprocessor. For example, a conductive yarn may comprise a yarn treated with one or more metals to impart conductive properties, such as by dipping, spraying, vapor deposition, vacuum evaporation, sputtering, magnetron sputtering, ion plating, and autocatalytic electroless plating processes and other processes known in the art. In autocatalytic processes, a yarn is knitted into a structure, many structures are then plated with a metal during the electroless process, and then de-knitted, leaving the yarn. The yarn is then plated with the metal, which is then knitted into, for example, a glove. Many natural and synthetic yarns are suitable for the conductive yarn, including nylon, polyester, cotton, rayon, and like materials and blends thereof. In some embodiments, the natural and synthetic yarns are treated with metals, such as, but not limited to, silver, gold, aluminum, nickel, tin, stainless steel, copper, carbon, or combinations thereof, and metal alloys such as aluminum-copper, aluminum-magnesium, copper-gold, copper-nickel, copper-palladium, gold-palladium, gold-silver, iron-nickel and silver-palladium, or combinations thereof.

In some embodiments, a conductive yarn may be plaited into the fingertips only or, alternatively, the entire glove. In yet other embodiments, irrespective of whether the liner comprises a plaited conductive yarn or a single layer incorporating more than one yarn, fewer than all five fingers may contain a conductive yarn, for example, only the thumb, index, and middle fingertips may comprise a conductive yarn. In some embodiments according to the invention, it may be desirable to knit a conductive yarn in the fingertips and the crotch extending between the index finger and the thumb, the crotches between other fingers, or in other areas of the hand susceptible to contact with moving parts. For example, some devices or tools, such as table saws, have metal components which carry an electrical signal. In the event a conductive element contacts the metal component, a change in conductivity results. This change can be used to activate a safety system, for example, a braking system, disabling the device or tool and preventing further motion of, for example, a saw blade. The use of conductive gloves with such tools results in fewer and less serious injuries to users, particularly where the gloves are knitted with a cut-resistant yarn, such as KEVLAR® as well as conductive yarn to form at least one conductive region. Tools having such features are disclosed in US Patent Publ. No. 2011/0061769 and U.S. Pat. No. 8,006,595, each of which is hereby incorporated by reference in its entirety. It may also be desirable to include conductive yarns in regions or entirety of other knitted clothing, such as aprons, shirts, ties, and the like, that may become caught inadvertently in machines, such as two-roll mills, conveyors, industrial mixers, and the like, as well as saws, drills, and the like.

The stretchable force detecting sensors 140, according to embodiments of the disclosure. For example, a stretchable force detecting sensor 140 may be sandwiched between an encapsulant, which may be overprinted or coated onto the stretchable sensor 140, and a stretchable conductor. The stretchable sensor 140 is then laminated with a thermoplastic material, such as a thermoplastic polyurethane material, onto a film or fabric. In at least one embodiment, the fabric is the inner glove 100 or inner glove 200 or inner glove 300 described above. A stretchable sensor 140 may be washed, wherein the life of the inner glove 100, 200, 300 is extended. In at least one embodiment, the fabric is an outer glove, such as a knitted glove similar to, for example, the models GT3279, GT3280, GT3287, or GT3440 gloves marketed by Ansell Ltd.

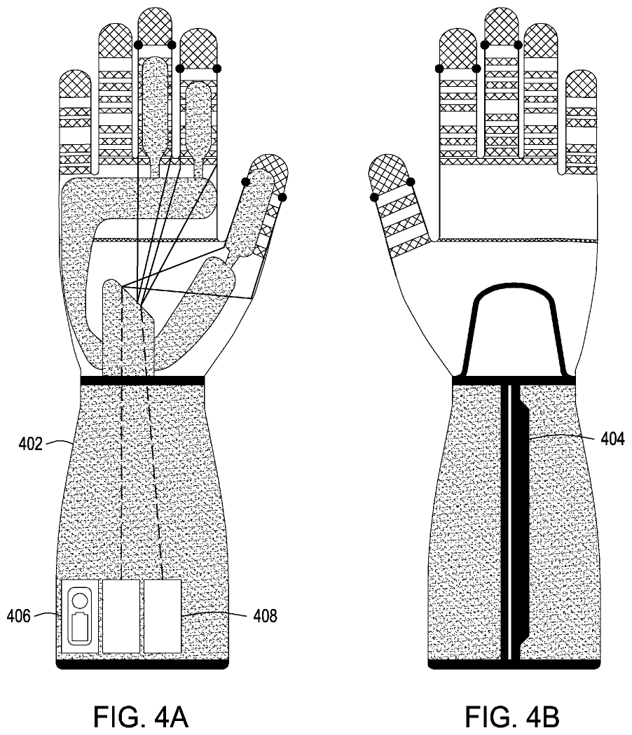

FIGS. 4A and 4B depict the palm and backhand views of FIG. 1A and FIG. 2, respectively, along with a robotic sleeve portion 402 attached to the cuff, in accordance with some embodiments of the disclosure. The robotic sleeve portion 402 may be attached to the cuff 114 using hook and loop type fasteners or other suitable means. In some embodiments, the robotic sleeve portion 402 includes a hook and loop fastener 404 to secure the sleeve about a person's wrist or forearm. In some embodiments, the robotic sleeve portion 402 includes a controller 406, also referred to as a control unit, and the tendon actuators and motors 408 that together, control movement of the tendons 150. The one or more actuators 408 may be radial actuator including a radial winding spool for spooling the tendons 150. In some embodiments, the controller 406 can include at least one CPU or microprocessor, at least one memory storage, at least one input to receive information and communications signals including force detection signals from the force detectors 140, and at least one output to transmit information and communication signals. The controller 406 may include wireless data capabilities such as BLUETOOTH, WIFI, NFC and the like for transmitting and receiving data. The controller memory may store user usage statistics and programs that when executed, cause the actuators and tendons to perform specific functions. In some embodiments, one or more controllers may be in electrical communication with the at least one force detecting sensor 140 and the at least one actuator 408, wherein the at least one tendon 150 undergoes a tensile force pulling the at least one finger or thumb in a closed position upon detection of a force by the at least one force detecting sensor to enhance the hand strength of a user.

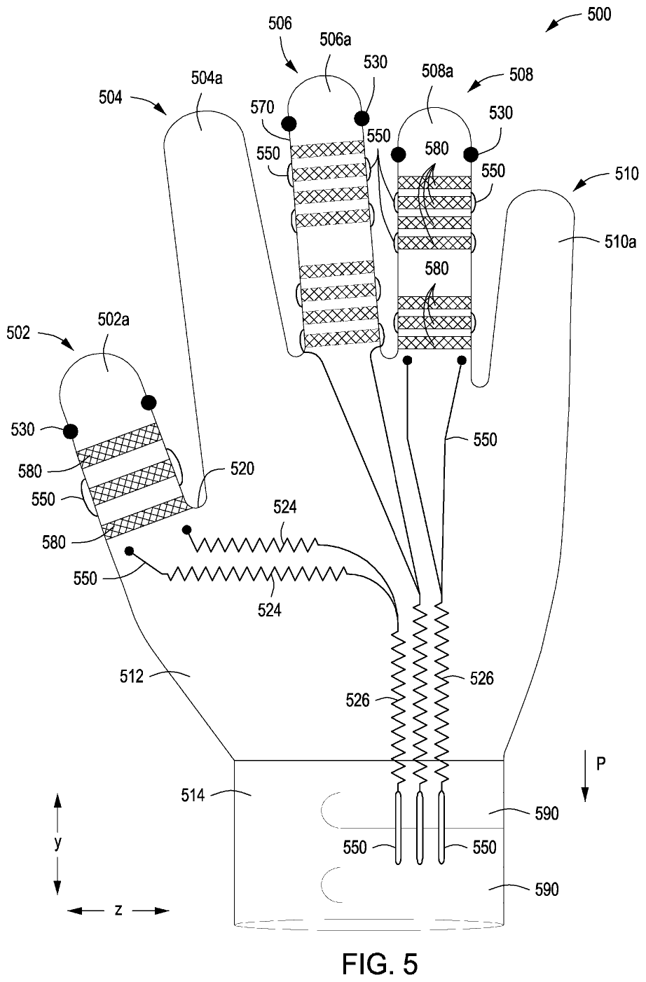

FIG. 5 depicts a palm side of a thin, flexible, knitted left-handed inner glove 500 having a modified cuff and tendons, according to some embodiments of the disclosure. FIG. 5 depicts a palm side of a thin, flexible, knitted left-handed inner glove 500 having tendons 550, according to some embodiments of the disclosure. As seen in FIG. 5, the inner glove 500 is a left-handed inner glove in which the palm 512 is displayed. The inner glove 500 includes a thumb 502, an index finger 504, a middle finger 506, a ring finger 508, a little finger 510, a modified cuff 514, and an opening (not shown) capable of receiving the hand of a wearer. The modified cuff 514 may be constructed such that materials comprising the modified cuff 514 stretch in an x direction (lateral) and do not stretch in a y direction (longitudinal), or stretch longitudinally in lesser amounts than in the x direction, to aid in a compression fit and stabilization of forces applied by the tendons 550. The modified cuff 514 combines a raised portion above a natural wrist line of a wearer, and a natural wrist portion below the natural wrist line. The raised portion of the modified cuff 514 has no or minimal stretch in x and y directions for stabilization of forces. The natural wrist portion of the modified cuff 514 will either have minimal stretch similar to the raised portion, or else will allow stretch in y-direction for improved fit in wrist area. The design of the modified cuff 514 may further comprise an integrated thumb strap, which “floats” along the back of the thumb area and allows the tendons 550 to be interlaced on both sides of the thumb areas, allowing a directional pull when the tendons 550 are activated.

The tendons 550 are housed within tendon channels 524. As shown, the tendon 550 for the thumb 502 traverse through the lateral tendon channels 524, across the palm 512 in a lateral direction and then into longitudinal tendon channels 526. The tendons 550 for the middle finger 506 and the ring finger 508 traverse through the longitudinal tendon channels 526. The longitudinal channels 526 are disposed within the palm 512 and the modified cuff 514. As shown, there are two lateral tendon channels 524 for housing one tendon 550 for the thumb 502. The lateral tendon channels 524 are disposed near the crotch areas 520 of the thumb 502. The lateral tendon channels 524 are approximately 1-2 mm in width for housing the tendon 550. The lateral tendon channels 524 are approximately 20-60 mm in length. Also, as shown, there are three longitudinal tendon channels 526, one each for the tendons 550 for the thumb 502, the middle finger 506, and the ring finger 508. The longitudinal tendon channels 526 are approximately 3 mm in width for housing the tendons 550 and are approximately 100-150 mm in length. The lateral tendon channels 524 and the longitudinal tendon channels 526 may be knitted into a fabric of the inner glove 550.

Each of the thumb 502, the index finger 504, the middle finger 506, the ring finger 808, and the little finger 510 further comprises tips at distal ends, a thumbtip 502a, an index fingertip 504a, a middle fingertip 506a, a ring fingertip 508a, and a little fingertip 510a. The inner glove 500 also comprises tendons 550. As shown, the thumb 502, the middle finger 506, and the ring finger 508 each comprise the tendon 550. In practice, there can be a tendon 550 in any one of each of the thumb 502, and the fingers 504, 506, 508, and 510 or any combination thereof. As above, the tendons 550 can be a yarn, a string, a polymer, a wire, a fiber, or any monofilament. In at least one embodiment, the tendon 550 is a monofilament comprising materials and/or coating, as discussed above. In practice, many knitted gloves marketed by Ansell Ltd. can have the tendons 550 added to manufacture the inner glove 500. Also, at least one inner glove 500 comprises one or more hooks and loops fastener strap 590 for securing the inner glove 500 to an outer glove, as described more fully below. As shown, there are two straps 590.

As shown, the tendons 550 are adhered or otherwise tied to a fingertip or thumbtip, such as thumbtip 502a, the middle fingertip 506a and the ring fingertip 508a at knots 530. As shown, the tendon 550 is interlaced within, for example, the zonal bands 580. In other words, the tendons 550 enter through the knitted structure of the middle finger 506. The tendon 550 stays inside the middle finger 506 before exiting at a plurality of instances corresponding with zonal bands 580. As shown, the tendons 550 traverse a lateral perimeter 570 of the middle finger 506. As will be described more fully below, providing a pulling force P at an inflection point of the tendon 550, significantly similarly as above, causing a bending of the middle finger 506.

The zonal bands 580 may comprise fortified areas, e.g., reinforcements, for example, reinforcements having variable knit plaiting yarns to strengthen the areas around the ingress and egress of the tendons 550 into the glove. The zonal bands 580 are made stiffer and stronger, allowing a stronger pulling force to be applied without damaging the inner glove 500. In some embodiments, the zonal bands 580 can be reinforcements made by Knitted Variable Stitch Dimension (KVSD), as discussed above, as is described in U.S. Pat. No. 7,434,422, which is commonly assigned and incorporated by reference in its entirety. The inner glove 500 may also comprise two or more yarns, seamless knit technology according to the co-pending, commonly assigned U.S. Patent Publ. Nos. 2010/0275341 and 2010/0275342, each of which is herein incorporated by reference in its entirety. The inner glove 500 may also comprise a knitted layer incorporating Automated Knitted Liner (AKL) technologies, developed by Ansell Limited. The inner glove 500 may also comprise a knitted layer having two or more yarns having variable plaiting, as is known to those in the art, to make an inner glove 500 having borders zonal bands 580. At least one embodiment according to the present disclosure includes at least one of three ridge reinforced gloves, light duty, medium duty, and heavy duty gloves, as disclosed in US Publ. No. 2013/0205469, which is commonly assigned and incorporated herein by reference in its entirety. Those ridge-reinforced gloves comprise ridges. Accordingly, the ridge can be used as the zonal bands 580 and the ridge-reinforced gloves can be used as the inner glove 500.

The inner glove 500 may have an outer glove, such as the outer glove 200, as shown above, releasably attached thereto. Also, embodiments described within the disclosure also contemplate a middle layer glove (not shown) disposed between the inner glove 500 and the outer glove 200. A middle layer glove may, optionally, be a very thin glove optionally attached to a cuff of the inner glove 500. The inner glove 500 comprises sensors for sensing forces applied by the fingers of a wearer of the inner glove 500, which in turn are in communication with actuators, as described above. One such sensor is Interlink Electronics' FSR™ 400 series, which is part of the single zone Force Sensing Resistor™ family. Force sensing resistors, or FSRs, are robust polymer thick film (PTF) devices that exhibit a decrease in resistance with increase in force applied to the surface of the sensor. This force sensitivity is optimized for use in human touch control of electronic devices, such as many tools used in automotive electronics, medical systems, and in industrial and robotics applications. A sensor FSR402 model is at least one suitable sensor. Also, at least one sensor includes elastic sensors, as described above. At least one exemplary embodiment of the disclosure includes an elastic sensor comprising a dielectric elastomer. For example, at least one dielectric elastomer sensor comprises an elastic film that is coated with flexible electrodes. Such sensors measure electrical capacitance differences during deformation or tensile/compressive forces. Moreover, elastic sensors comprising a dielectric elastomer may be washed. Many other sensors, such as those listed above, are also suitable.

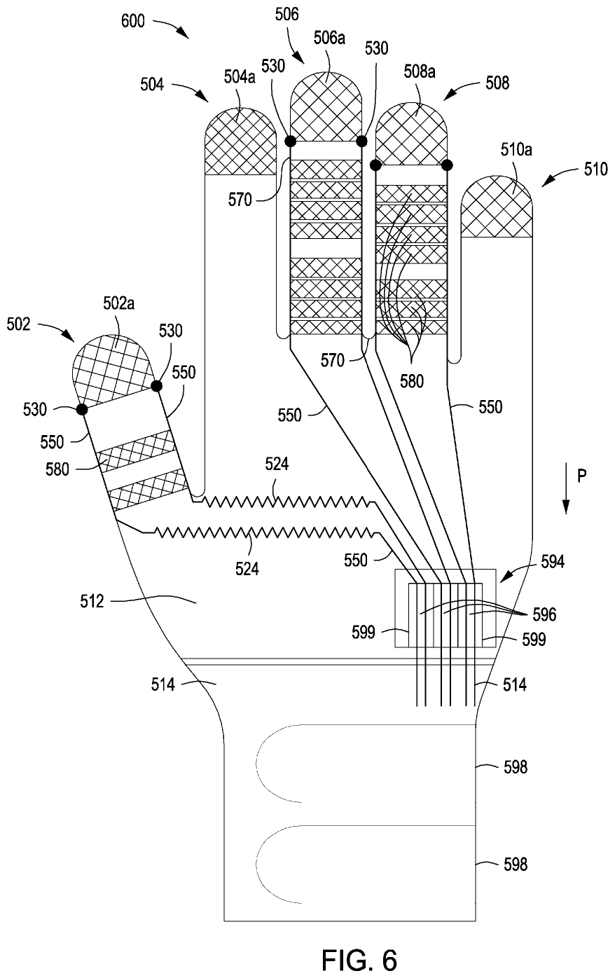

FIG. 6 depicts a palm side of a thin, flexible, knitted left-handed inner glove 600 having a modified cuff 514, tendons 550, and a stabilizer 594 having tunnels 596, according to some embodiments of the disclosure. As seen in FIG. 6, the inner glove 600 is a left-handed inner glove in which the palm 512 is displayed. The inner glove 600 includes a thumb 502, an index finger 504, a middle finger 506, a ring finger 508, a little finger 510, a modified cuff 514, and an opening (not shown) capable of receiving the hand of a wearer. The modified cuff 514 may be constructed such that materials comprising the modified cuff 514 stretch in an x direction (lateral) and do not stretch in a y direction (longitudinal), or stretch longitudinally in lesser amounts than in the x direction, to aid in a compression fit and stabilization of forces applied by the tendons 550. The modified cuff 514 optionally combines a raised portion above a wrist line of a wearer, and a wrist portion below the natural wrist line, i.e., adjacent to a cuff 514 of the inner glove 600. The raised portion of the modified cuff 514 has no or minimal stretch in x and y directions for stabilization of forces. The wrist portion of the modified cuff 514 will either have minimal stretch similar to the raised portion, or else will allow stretch in y-direction for improved fit in wrist area. The design of the modified cuff 514 may further comprise two integrated thumb straps 598 to tighten the inner glove 600 with an outer glove (not shown).

The tendons 550 are housed within tendon channels 524 that emanate from the thumb 502. As shown, the tendon 550 for the thumb 502 traverse through the lateral tendon channels 524, across the palm 512 in a lateral direction and then into longitudinal tendon channels 526. The lateral tendon channels 524 are disposed near the crotch areas 520 of the thumb 502. The tendons 550 are interlaced into and out of the inner glove 600 at the zonal bands 580, allowing a directional pull P when the tendons 550 are activated, which is substantially co-axial with a longitudinal axis of the inner glove 600.

The lateral tendon channels 524 are approximately 1-2 mm in width for housing the tendon 550. The lateral tendon channels 524 are approximately 20-60 mm in length. The longitudinal tendon channels 524 are approximately 3 mm in width for housing the tendons 550 and are approximately 100-150 mm in length. The lateral tendon channels 524 may be knitted into a fabric of the inner glove 600. The tendons 550 for the middle finger 506 and the ring finger 508 traverse to the stabilizer 594 and through the channels 596. The stabilizer 594 may be screenprinted on the inner glove 600. The stabilizer 594 may be a patch sewn onto the inner glove 600, wherein the channels 596 are formed by longitudinal stitching 599.

Each of the thumb 502, the index finger 504, the middle finger 506, the ring finger 808, and the little finger 510 further comprises tips at distal ends, a thumbtip 502a, an index fingertip 504a, a middle fingertip 506a, a ring fingertip 508a, and a little fingertip 510a. The inner glove 500 also comprises tendons 550. As shown, the thumb 502, the middle finger 506, and the ring finger 508 each comprise the tendon 550. In practice, there can be a tendon 550 in any one of each of the thumb 502, and the fingers 504, 506, 508, and 510 or any combination thereof. As above, the tendons 550 can be a yarn, a string, a polymer, a wire, a fiber, or any monofilament. In at least one embodiment, the tendon 550 is a monofilament comprising materials and/or coating, as discussed above. In practice, many knitted gloves marketed by Ansell Ltd. can have the tendons 550 added to manufacture the inner glove 600. Also, at least one inner glove 600 comprises one or more hooks and loops fastener strap 598 for securing the inner glove 600 to an outer glove, as described more fully below. As shown, there are two straps 598, which provide a compression fit in the cuff 514 area of the glove.

As shown, the tendons 550 are adhered or otherwise tied to a fingertip or thumbtip, such as thumbtip 502a, the middle fingertip 506a and the ring fingertip 508a at knots 530. As shown, the tendon 550 is interlaced within, for example, the zonal bands 580. In other words, the tendons 550 enter through the knitted structure of the middle finger 506. The tendon 550 stays inside the middle finger 506 before exiting at a plurality of instances corresponding with zonal bands 580, i.e., the tendons are interlaced. As shown, the tendons 550 traverse a lateral perimeter 570 of the middle finger 506. As will be described more fully below, providing a pulling force P at an inflection point (not shown) of the tendon 550, significantly similarly as above, causing a bending of the middle finger 506, the ring finger 508, and the thumb 502.

The zonal bands 580 may comprise fortified areas, e.g., reinforcements, for example, reinforcements having variable knit plaiting yarns to strengthen the areas around the ingress and egress of the tendons 550 into the glove 600. The zonal bands 580 are made stiffer and stronger, allowing a stronger pulling force to be applied without damaging the inner glove 600. In some embodiments, the zonal bands 580 can be reinforcements made by Knitted Variable Stitch Dimension (KVSD), as discussed above, as is described in U.S. Pat. No. 7,434,422, which is commonly assigned and incorporated by reference in its entirety. The inner glove 600 may also comprise two or more yarns, seamless knit technology according to the co-pending, commonly assigned U.S. Patent Publ. Nos. 2010/0275341 and 2010/0275342, each of which is herein incorporated by reference in its entirety. The inner glove 600 may also comprise a knitted layer incorporating Automated Knitted Liner (AKL) technologies, developed by Ansell Limited. The inner glove 600 may also comprise a knitted layer having two or more yarns having variable plaiting, as is known to those in the art, to make an inner glove 600 having borders zonal bands 580.

At least one embodiment of the disclosure comprises a middle glove, which can be any knitted liner. The middle glove is sewn onto the inner glove 100, 200, 300, 500, 600 at the wrist. The middle glove further comprises a sewn-on tab or extension which is used to create the tendon/wiring tunnel so that the tendons 180, 550 travel from the palm area of the inner glove 100, 200, 300, 500, 600 and out the cuff 514 of the inner glove for connection with the actuators. In at least one embodiment, the middle glove has channels sewn into the middle glove, substantially similar to the longitudinal stitching 599 discussed above. The middle glove is sewn up one side of the “tunnel” extension, around the side and back at the wrist line, and then down the other side of the tunnel extension to close the assembly and create the tunnel. The attachment method to the inner glove 100, 200, 300, 500, 600 is unique in that it allows the inner glove, such as the inner glove 600 and the cuff 514, to be fully constructed before any tendons 180, 550 or wires are attached. The middle glove is integrated fully as the overlay for the inner glove 100, 200, 300, 500, 600 to improve don/doff and protect the tendons 180, 550 and electronics, i.e., sensors during use and when the outer glove is being replaced. This creates a layered inner glove once fully assembled. The outer glove functions to protect the inner glove 100, 200, 300, 500, 600 and improve grip and/or durability for the user. In at least one embodiment of the invention, the outer glove comprises a dual-window clasp. The strap(s) of the inner gloves 100, 200, 300, 500, 600 can be engaged with the outer glove.

The term “flexing” or “flex” refers to finger movements, such as bending fingers and/or fingertips, making a fist, gripping, grasping, clenching or otherwise folding the fingers. As disclosed herein, the term fingertips of a glove may be defined as the distal end of a fingerstall or thumbstall of a glove. A glove herein comprises one or more stalls for a finger and/or a thumb and/or defines an inner volume for receiving a hand or parts of a hand. Integrally formed means an article that, once formed, cannot be disassembled without causing the destruction of the article, such as the gloves in the present application

In some embodiments, the liner comprises a polymeric or elastomeric material disposed thereon as a coating. Typical polymeric or elastomeric materials that may be used include natural rubbers, synthetic rubbers, natural isoprene, synthetic isoprene, guayule, butadienes, styrene-butadienes, nitrile-butadienes, carboxylated nitrile-butadienes, poly (vinyl chloride), polyurethane, polychloroprene, and blends, mixtures thereof, and the like. Polymeric and elastomeric materials that are particularly useful, in that they provide, instead of an insulative effect as nearly all polymeric and elastomeric materials do, and rather approach the conductivity of dielectric materials, are, for example, nitrile-butadiene rubbers. The polymeric or elastomeric emulsion composition may comprise ingredients such as surfactants, defoamers, pigments, colorants, plasticizers, thixotropic agents, shear thickeners, shear thinners, processing aids, fillers, and the like, as known to those of ordinary skill in the art. Some additives, such as carbon, carbon fiber, carbon nanotubes and the like are suitable conductive materials that provide conductive properties.

A glove with a polymeric or elastomeric coating is disposed on a liner by dipping, spraying, screenprinting, or otherwise painting processes. Dipping processes include a fingertip dip, a palm dip, a three-quarters dip, a full dip, or a dip covering the backhand and knuckles without a coating disposed on the fingertips, resulting in different levels and regions of protection, any of which may be appropriate for a given application. In another embodiment, the finger and palm areas may be coated, while the backhand may be uncoated. Also, the screenprinted stabilizer 594 may comprise a polymeric emulsion, dispersion, or suspension of natural or synthetic elastomer molecules.

The terms “emulsion,” “dispersion,” and “suspension” are generally analogous and indicate a system in which small particles of a substance, such as rubber particles, are mixed with a fluid (such as water and/or alcohols and/or other organic fluids) but are at least partially undissolved and kept dispersed by agitation (mechanical suspension) and/or by the molecular forces in a surrounding medium (colloidal suspension). Any embodiment of the invention contemplated herein may further comprise emulsions having typical and suitable components for rubber or elastomeric formulations, such as accelerators, such as guanidines, thiazoles, thiurams, sulfenamids, thioureas, dithiocarbamates, and xanthanates. Emulsions according to embodiments of the invention may further comprise surfactants, such as sodium dodecyl sulfates and polyvinyl alcohols, activators, such as zinc oxides, cross-linking agents and curatives, such as elemental sulfur and/or polysulphidic donors, such as xanthogens, such as dibutyl xanthogen disulfides and/or diisopropyl xanthogen disulfides. The emulsions contemplated herein may also comprise other additives, such as anti-oxidants, anti-ozonants, rheology-modifiers and thickening agents, such as various clays and aluminosilicates, pH adjusters, such as hydroxides, such as potassium hydroxide and/or ammonium hydroxide, pigments, processing agents, waxes/lubricating agents, and/or fillers as are known to those in the art.

The term “polymer” generally includes, but is not limited to, homopolymers, copolymers, such as for example, block, graft, random and alternating copolymers, terpolymers, etc., whether branched or linear polymers, and blends and modifications thereof. Furthermore, unless otherwise specifically limited, the term “polymer” includes all possible geometrical configurations of the molecule, including, but are not limited to, isotactic, syndiotactic and random symmetries.

The term “thermoplastic” generally includes polymer materials that become reversibly pliable, moldable, and heatable above a specific temperature and solidify upon cooling. The term “thermoset” generally includes are polymer materials that strengthen following heating and solidification, but cannot be successfully remolded or reheated after an initial heat-forming due to cross-linking. The term “thermoplastic elastomer” (TPE) are a class of copolymers comprising both thermoplastic and elastomeric/thermoset materials properties and generally have crosslinking between adjacent polymeric molecular chains, generally allowing materials made therefrom to be re-heatable. The term “rubber” generally indicates elastomers produced from natural rubber latexes or synthetic elastomers.

Exemplary thermoplastics include, without limitation, polychloroprenes, butyl rubbers, natural rubber, synthetic polyisoprenes, poly(vinyl) chlorides, polyesters, polyamides, polyfluorocarbons, polyolefins, polybutadienes, polyurethanes, polystyrenes, poly(vinyl) alcohols, and copolymers of the foregoing, and elastomeric polymers such as elastic polyolefins, copolyether esters, polyamide polyether block copolymers, block copolymers having the general formula A-B-A′ or A-B, such as nitrile-butadiene rubber (NBR), styrene-poly(ethylene-propylene)-styrene, styrene-poly(ethylene-butylene)-styrene, (polystyrene/poly(ethylene-butylene)/polystyrene, poly(styrene/ethylene-butylene/styrene), copoly(styrene/ethylene-butylene), A-B-A-B tetrablock copolymers and the like and/or blends of any of the foregoing and/or any of the foregoing having additional chemical groups, molecules, atoms or other moieties substituted thereon, i.e., derivatives of any of the foregoing thermoplastics.

In some embodiments, a foamed polymeric or elastomeric coating comprises a microporous structure, for example, the foamed emulsion may contain approximately 10%, 20%, 30%, 40% or more aeration. A foamed polymeric or elastomeric coating may be disposed on an inner, middle or outer glove, as disclosed herein. Once the emulsion is foamed with the right air content and the viscosity is adjusted, refinement of the foam is undertaken by using the right whipping impeller stirrer driven at an optimal speed first and the air bubble size adjusted using a different impeller run at a reduced speed. Foams and methods for producing foams having different amounts of bubbles, and differing bubble sizes, are disclosed in commonly-assigned U.S. Pat. No. 8,137,606, which is herein incorporated by reference in its entirety. The polymeric or elastomeric emulsion may comprise surfactants to stabilize the foamed emulsion. The emulsion quickly gels on a liner because of the action of a coagulant, as discussed below, resident on the surfaces of the yarns, forming chocking regions between the fibers preventing further entry of the foamed latex emulsion into the thickness of the knitted liner. Gloves having foamed and unfoamed features are disclosed in commonly-assigned U.S. Patent Publ. No. 2010/0275341 and 2010/0275342, each of which is herein incorporated by reference in its entirety.

As mentioned above, the polymeric or elastomeric coating may optionally be foamed to provide a more flexible glove providing protection against physical wear as well as protecting the wearer from chemicals, oils, acids, and the like. For example, increasing the air cell content reduces the modulus of elasticity of the polymeric or elastomeric coating, thereby increasing the flexibility of the glove. Some embodiments of the invention comprising a foamed latex coating may use a suitable combination of a surfactant, control of air content in the foamed coating, control of the viscosity of the elastomeric lattices, and suitable former temperatures for dipping processes. Methods for disposing a foamed coating on a liner can include those of commonly assigned U.S. Pat. No. 7,814,571, herein incorporated by reference in its entirety. In some embodiments, the foamed coating may be cured and further post-treated with a chlorination process to provide a lubricious surface on the coating of the glove.

Any numerical values recited herein are exemplary, are not to be considered limiting, and include ranges therebetween, and can be inclusive or exclusive of the endpoints. Optional included ranges can be from integer values therebetween, at the order of magnitude recited or the next smaller order of magnitude. For example, if the lower range value is 0.1, optional included endpoints can be 0.2, 0.3, 0.4 . . . 1.1, 1.2, and the like, as well as 1, 2, 3 and the like; if the higher range is 10, optional included endpoints can be 7, 6, and the like, as well as 7.9, 7.8, and/or the like.

Although some embodiments have been discussed above, other implementations and applications are also within the scope of the following claims. Although various embodiments herein have been referred to with particularity, it is to be understood that these embodiments are merely illustrative of the principles and applications of the various embodiments. It is therefore to be understood that modifications may be made to the illustrative embodiments and other embodiments may be devised without departing from the spirit and scope of the present disclosure.

Publications and references, including but not limited to patents and patent applications, cited in this specification are herein incorporated by reference in their entireties as if each individual publication or reference were specifically and individually fully set forth herein. Any patent application to which this application claims priority is also incorporated by reference herein in the manner described above for publications and references.

1. A glove system capable of assisting a user, comprising:
a first knitted glove having at least one finger or thumb having at least one force detecting sensor and a cuff,
at least one tendon adhered to a portion of at least one finger or thumb and traversing a length of the at least one finger or thumb to the cuff, wherein the at least one tendon is interlaced within at least one reinforced zonal band disposed along the at least one finger or thumb;
at least one actuator attached to the at least one tendon; and
at least one controller in electrical communication with the at least one force detecting sensor and the at least one actuator, wherein the at least one tendon undergoes a tensile force pulling the at least one finger or thumb in a closed position upon detection of a force by the at least one force detecting sensor.

2. (canceled)

3. The glove system of claim 1, further comprising tendons comprising a monofilament.

4. The glove system of claim 3, the monofilament further comprises a p-aramid, m-aramid, polyester, fiberglass, ultra-high molecular weight polyethylene, and blends thereof.

5. The glove system of claim 1, wherein the at least one finger and at least one thumb comprise differing stitch setups to produce variable stitch dimensions.

6. The glove system of claim 1, wherein the at least one actuator is a radial actuator including a radial winding spool for spooling the at least one tendon.

7. The glove system of claim 1, further comprising a second knitted glove disposed over the first knitted glove.

8. The glove system of claim 7, further comprising a middle glove disposed between the second knitted glove and the first knitted glove.

9. The glove system of claim 7, further comprising a middle glove between the second knitted glove and the first knitted glove and sewn to the first knitted glove.

10. The glove system of claim 1, further comprising sensors having an encapsulant disposed on an exposed side and/or a side opposite the exposed side.

11. The glove system of claim 1, further comprising sensors having a non-stretchable conductor.

12. The glove system of claim 1, further comprising sensors having a stretchable conductor.

13. The glove system of claim 1, further comprising knitted or sewn reinforcements within the first knitted glove.

14. The glove system of claim 1, further including at least one dielectric elastomer sensor comprising an elastic film that is coated with flexible electrodes.

15. The glove system of claim 1, wherein the tendon is interlaced within a plurality of regions comprising knitted reinforcements.

16. A glove system capable of assisting a user, comprising:
a first knitted glove having at least one finger or thumb having at least one force detecting sensor and a cuff,
at least one tendon adhered to a portion of at least one finger or thumb and traversing a length of the at least one finger or thumb to the cuff; and
at least one actuator attached to the at least one tendon,
wherein the at least one force detecting sensor is in electrical communication with at least one conductive member that is in electrical communication with a controller having a microprocessor, and wherein the microprocessor determines a force that is applied to the at least one tendon and configured to pull the at least one finger or thumb in a closed position.

17. The glove system of claim 16, wherein the controller is configured to transmit the determined force to the at least one actuator or a power supply that supplies power to the at least one actuator.

18. The glove system of claim 16, wherein the at least one conductive member is a plurality of conductive yarns to electrically connect the at least one force detecting sensors to the controller.

19. The glove system of claim 16, wherein the at least one tendon is interlaced within at least one reinforced zonal band disposed along the at least one finger or thumb.

20. The glove system of claim 19, wherein the at least one reinforced zonal band disposed along the at least one finger or thumb is formed using Knitted Variable Stitch Dimension (KVSD) technology.
